(19) United States
(12) Patent Application Publication
(10) Pub. No.: US 2010/0151442 A1
(43) Pub. Date: Jun. 17, 2010

(54) METHOD FOR DETECTING EMERGING PANDEMIC INFLUENZA

(76) Inventors: Niveen M. Mulholland, Rockville, MD (US); Joseph A. Bogan, JR., Germantown, MD (US); Ellen Petrangelo, Gaithersburg, MD (US); Nicole M. Waybright, Silver Spring, MD (US); Peggy T. Lowary, Frederick, MD (US)

Correspondence Address: HUSCH BLACKWELL SANDERS LLP, 4801 Main Street, Suite 1000, KANSAS CITY, MO, 64112, US

(21) Appl. No.: 12/332,847

(22) Filed: Dec. 11, 2008

(51) Int. Cl. C12Q 1/70

(52) U.S. Cl. 435/5

(57) ABSTRACT

A method for detecting emerging pandemic influenza strains is provided. RT-PCR is used to detect HPAI followed by pyrosequencing to detect codons defining human or avian influenza signatures. This method screens for avian influenza viruses containing mutations suspected of making the virus more infective or virulent to humans.

### BACKGROUND OF THE INVENTION

The world is currently in a Pandemic Alert Period. A pandemic due to avian influenza would result if the current form of an avian virus were to mutate to be infective to humans, since humans would lack immunity for this new strain. Avian Influenza Virus (AIV) has already crossed from poultry to human and in isolated situations from human to human.

Influenza virus is a member of the orthomyxoviridae family, and is a single stranded RNA virus with a segmented genome. Influenza A is responsible for seasonal flu and is the most virulent human pathogen of the three subtypes. Influenza B can cause illness in humans but does not mutate, so most of the population develops immunity. Influenza C is very rare and usually only results in mild illness.

New strains of Influenza A virus emerge through genetic drift and genetic shift. Genetic drift is caused by mutations in the RNA genome during replication of the viral RNA. The result is a protein with an altered amino acid sequence. Genetic shift, or genetic reassortment, is the exchange of gene segments between influenza viruses. Genetic shift can occur naturally when two or more different viruses infect the same host, resulting in the emergence of new subtypes.

Influenza A proteins have at least 52 amino acid sites shown to be specific to viruses which infect humans. For example, genetic mutations yielding an H5N1 strain are highly virulent and/or infective to humans and present a significant public health threat. The currently circulating H5N1 virus has a high fatality rate in infected humans, typically greater than 60%. Fortunately only a small number of infected individuals have been reported to date. The currently limited human to human transmission is attributed to inefficient viral infection and propagation in humans. However, in the case of a pandemic, screening capabilities will be a critical first step for controlling the continual spread of disease. Current surveillance of influenza strains that threaten the human population involves simple identification of the presence of the strain. There is no attempt to distinguish between avian-specific and human-specific viruses.

There is therefore a need to develop a system that would efficiently detect mutations in the nucleotides encoding these amino acids and thus warn of an emerging threat. A rapid and targeted detection method for identifying mutations in the regions of the H5N1 virus which transition the virus to a more infective and virulent human strain would allow for detection of an emerging threat.

### SUMMARY OF INVENTION

In one illustrative aspect of the present invention there is provided a method comprising RRT-PCR and pyrosequencing to identify high pathogenic avian strains and then detect mutations in the high pathogenic avian strains that are indicative of a virus more infective to humans.

In another illustrative aspect of the present invention there is provided a method for detecting emerging pandemic influenza, the method comprising performing RRT-PCR to simultaneously detect multiple Influenza A virus subtypes while specifically detecting H5 strains; and pyrosequencing targeted regions of gene segments of the H5 strain to determine if critical human virulence signatures are present.

In still another illustrative aspect of the present invention, there is provided a method for detecting emerging pandemic influenza, the method comprising performing RRT-PCR to simultaneously detect multiple Influenza A virus subtypes while specifically detecting H5 strain; amplifying gene segments of the H5 strain; and pyrosequencing targeted regions of the H5 strain gene segments to determine if critical human virulence signatures are present.

In a further illustrative aspect of the present invention there is provided a method for detecting emerging pandemic influenza, the method comprising isolating virus RNA; performing RRT-PCR to simultaneously detect multiple Influenza A virus subtypes while specifically detecting H5N1 strain; amplifying gene segments of the H5N1 strain; pyrosequencing targeted regions of the gene segments of the H5N1 strain to determine if critical human virulence signatures are present; and conducting mutation analyses of the critical human virulence signatures.

### BRIEF DESCRIPTION OF THE SEVERAL VIEWS OF THE DRAWING

FIG. 1 is a flow chart illustrating the Sequencing for Avian Flu Epidemic method of the present invention.

FIG. 2 is an illustration of the eight segments of the Influenza A genome.

FIG. 3 is a chart illustrating the amino acids that are implicated in human virulence.

FIG. 4 is a comparison of human versus avian influenza genomic signature.

### DETAILED DESCRIPTION OF THE INVENTION

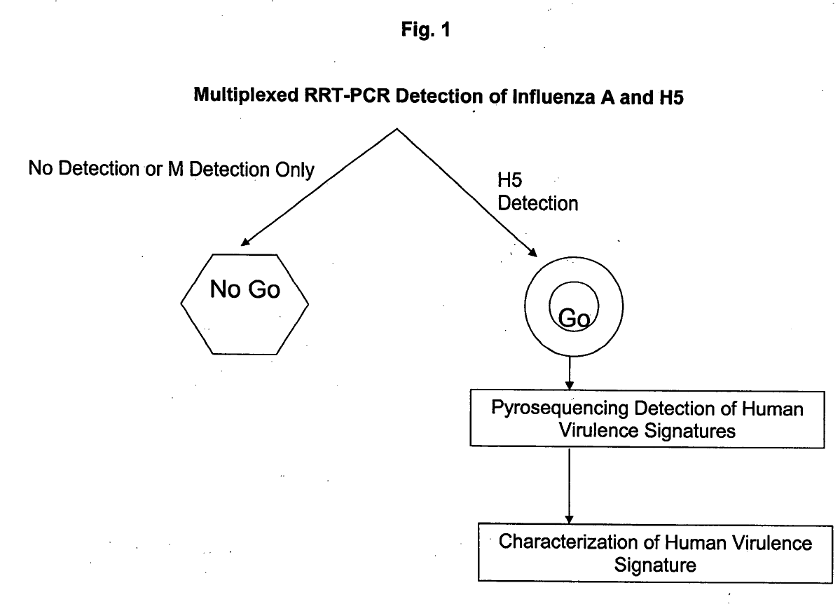

There is provided a method for detecting avian influenza virus and monitoring emerging mutations which pose a threat to the human population and would make the virus capable of causing pandemic. The method comprises sequencing two known methods, real time reverse transcription polymerase chain reaction (RRT-PCR) and pyrosequencing. The RRT-PCT step is used to identify Highly Pathogenic Avian Influenza (HPAI) strains. Once a HPAI strain has been identified, pyrosequencing is used to detect mutations that render the virus more infective to humans. This surveillance mechanism is designated herein as Sequencing for Avian Flu Epidemic, or SAFE, is illustrated in FIG. 1.

The SAFE method or system combines RRT-PCR and pyrosequencing technologies to detect current H5N1 AIV strains as well as emerging AIV threats that arise due to mutation. SAFE facilitates monitoring the global community for H5N1, and more importantly for mutations in H5N1 that render the virus more infective to humans. SAFE will provide data to establish a surveillance system which identifies sequence variations indicative of emerging influenza strains with greater human infectivity and virulence.

Pyrosequencing is used rather than more traditional sequencing platforms because of the increased speed with which exact sequence variation can be identified. Prior art methods to sequence avian influenza have been focused on sequencing the entire genome of isolated strains, providing the sequence libraries utilized herein. RRT-PCR has been used in the prior art to detect HPAI. However, there is no prior art method comprising sequencing these methods together to result in detection of mutations signaling greater human infectivity.

In addition to detecting emerging threats, the system provides public health officials with unique identifiers of the influenza strain posing the threat, which may assist vaccine developers and virologists in the pandemic response.

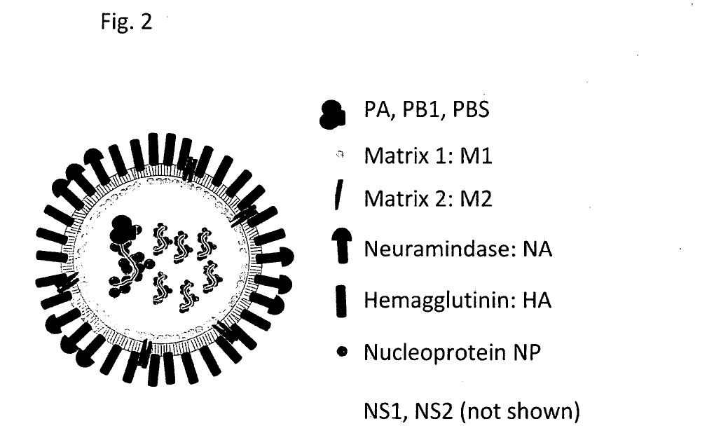

Avian Influenza Virus (AIV) is a single stranded RNA virus of the Influenza A family. The AIV genome, illustrated in FIG. 2, consists of eight individual segments. Each segment encodes for one or two viral proteins. The viral proteins give the virus its unique signature. Specifically, the hemaglutinin (HA) and neuraminidase (NA) surface proteins are responsible for viral nomenclature. For example, H5N1, the current avian virus, refers to an HA subtype 5 and NA subtype 1 combination. The M gene segment encodes for two proteins, M1 and M2. M2 is found in all Influenza A strains and is relatively invariant between strains.

For detection of trace levels of biological threats, PCR offers high selectivity and high sensitivity. In a preferred embodiment, the SAFE method utilizes RRT-PCR, resulting in high specificity and high sensitivity for the detection of viral RNA sequences. RRT-PCR allows simultaneous detection of all influenza A virus subtypes, targeting the invariant matrix gene (M) and for the specific detection of subtypes H5, H7 and H9, high pathogenic avian influenza strains.

In an illustrative example, the targeted subtype is H5, specifically H5N1. If H5N1 is detected, additional gene segments will be amplified and sequenced to determine if critical human virulence signatures are present. Complete mutation analyses will then be conducted within the targeted regions of the influenza genome that are implicated in human virulence. The sequenced data can then be screened against prior art sequence library to determine if mutation is present.

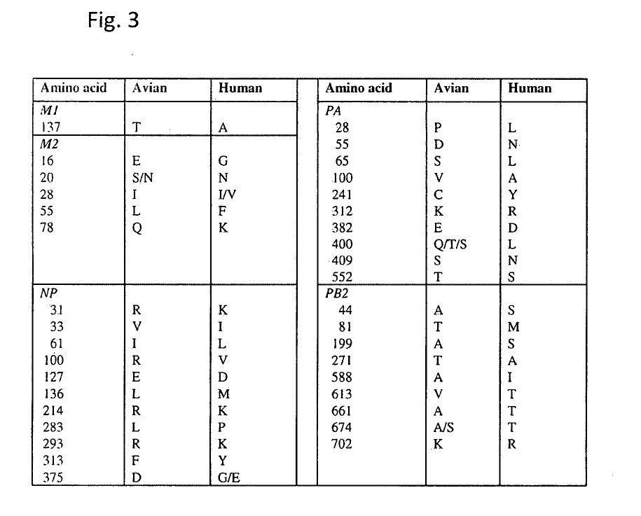

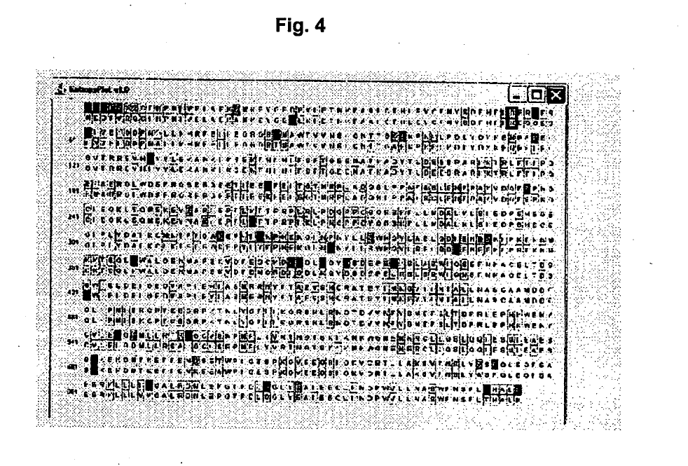

The presently preferred sequencing method is pyrosequencing. Pyrosequencing is a sequencing technology based on the iterative incorporation of specific nucleotides during primer-directed polymerase extension, providing real time sequence information. In the SAFE method, pyrosequencing if used to detect amino acid changes at the nucleotide level, i.e. codons, to distinguish human from avian influenza viruses. Prior art has generated position specific entropy profiles by comparing amino acid sequences of 95 avian and 306 human influenza strains. The analysis yielded 52 amino acids with entropy values less than −0.4, defined as conserved between human and avian viruses, as illustrated in FIG. 3. A comparison of human versus avian influenza genomic signature is illustrated in FIG. 4.

The following are illustrative, non-limiting embodiments of the present method. In one embodiment, the technician isolates and purifies viral RNA from swab or filter extracts using a viral RNA purification system. The RNA isolation may be conducted by any suitable method known to those skilled in the art, including method kits such as QiaAMP Viral RNA purification system. In the alternative, the RNA may be isolated and provided to the SAFE facility for further testing.

The next step is to multiplex the initial RRT-PCR influenza A/H5 screening step. RRT-PCR may be conducted in any suitable method known in the art. For convenience and economy, it is presently preferred to use a commercially available RRT-PCT kit. A suitable kit is the Taqman® One-Step RT-PCR Master Mix Reagents Kit by Applied Biosystems, Inc. For the initial screening step, PCR primers and probes specific for the matrix gene (M) and for hemaglutinnin subtype 5 gene (H5) are designed to allow for multiplexing. Results of large scale sequencing efforts included 534 primer sequences used for amplification and sequencing all eight gene segments. This set of established primers provides a valuable resource from which primer and probe sequences are extracted. An additional tool for primer/probe design is the NCBI Influenza Sequencing Database (http://www.ncbi.nlm.nih.gov/genomes/FLU/FLU.html). This database contains sequence information for all influenza strains that have been submitted to GenBank. To date, more than 30 k sequences have been included. Sequences from each subtype to be tested will be compared for commonalities. This will allow for determination of invariant sequences which will serve as priming sequences for both the PCR and the sequencing steps of the overall SAFE screening method.

Two real-time PCR detection chemistries that are suitable for the RRT-PCR step are fluorescent TaqMan® probes and SYBR Green I dye. TaqMan® assays are advantageous because very little optimization is required and multiplexing is readily accomplished by using specific probes with different fluorophores.

Features of the dsDNA intercalating dye SYBR Green I which make it a suitable method for this application include: a) similar levels of sensitivity as TaqMan®, b) fewer false negative than TaqMan® assays when detecting RNA viruses, c) successful transition from RRT-PCT using SYBR Green I detection to pyrosequencing has been reported d) disclosure of multiplexing strategies using SYBR Green I by utilizing melting curve analysis and e) SYBR Green I assays are significantly less expensive than TaqMan® assays.

The RRT-PCR data is then analyzed as is well known in the art for the presence of influenza virus subtypes, for example H5N1. If H5N1 is detected, additional gene fragments are amplified, and the biotinylated PCR product is purified on streptavidin coated beads, as is known in the art. Pyrosequencing is then used to determine the RNA sequence of potential human virulence sequences, by methods known in the art.

One of the major accomplishments of the SAFE method is it allows for rapid identification of the presence of H5N1 in a sample positive for influenza A, followed by the rapid identification of human virulence mutations. In an illustrative embodiment, the entire SAFE method, including RRT-PCR and pyrosequencing may be accomplished in as little as eight (8) hours. The materials required to perform the methods of the present invention may be packaged together to form a kit.

### EXAMPLES

### Example 1

### Primer and Probe Preparation

Primer and probe stocks need to be prepared under carefully controlled conditions to minimize any chance of contamination. Each primer probe mix will contain specific primers and probes for the target of interest as well as the water needed for the reaction. Each primer mix will contain specific primers for the target of interest. Master mix with enzymes and deoxyribonucleotide triphosphates (dNTPs), as well as buffers for the completion of the RRT-PCR reaction are added just prior to use.

Probes are ordered from Applied Biosystems, Inc., 850 Lincoln Center Drive, Foster City Calif. 94404, (www.appliedbiosystems.com). All probes are delivered in liquid format.

Primers are ordered from Integrated DNA Technologies (IDT), 1710 Commercial Park, Coralville Iowa 52241, (www.idtdna.com). Primers are received dry and stored at room temperature until reconstituted.

Using the tables below, mix components to create 20 reactions of primer probe mix that can be used at 10 μL per reaction for RRT-PCR:

TABLE 1
M3/H5

| | Final Concentration | |
|---|---|---|
| Component | Desired | Volume |
| 100 μM H5 F Primer | 300 nM | 3 μL |
| 100 μM H5 R Primer | 300 nM | 3 μL |
| 100 μM H5 Probe | 50 nM | 0.5 μL |
| 100 μM M3 F Primer | 900 nM | 9 μL |
| 100 μM M3 R Primer | 900 nM | 9 μL |
| 100 μM M3 Probe | 50 nM | 0.5 μL |
| TE | n/a | 175 μL |

TABLE 2
M3/H7

| | Final Concentration | |
|---|---|---|
| Component | Desired | Volume |
| 100 μM H7 F Primer | 300 nM | 3 μL |
| 100 μM H7 R Primer | 300 nM | 3 μL |
| 100 μM H7 Probe | 50 nM | 0.5 μL |
| 100 μM M3 F Primer | 900 nM | 9 μL |
| 100 μM M3 R Primer | 900 nM | 9 μL |
| 100 μM M3 Probe | 50 nM | 0.5 μL |
| TE | n/a | 175 μL |

TABLE 3
M3/H9

| | Final Concentration | |
|---|---|---|
| Component | Desired | Volume |
| 100 μM H9 F Primer | 300 nM | 3 μL |
| 100 μM H9 R Primer | 300 nM | 3 μL |
| 100 μM H9 Probe | 150 nM | 1.5 μL |
| 100 μM M3 F Primer | 900 nM | 9 μL |
| 100 μM M3 R Primer | 900 nM | 9 μL |
| 100 μM M3 Probe | 250 nM | 2.5 μL |
| TE | n/a | 175 μL |

TABLE 4
HNRPH1

| | Final | |
|---|---|---|
| | Concentration | |
| Component | Desired | Volume |
| 100 μM HNRPH1 F Primer | 300 nM | 15 μL |
| 100 μM HNRPH1 R Primer | 300 nM | 15 μL |
| 100 μM HNRPH1 Probe | 50 nM | 2.5 μL |
| TE | n/a | 967.5 μL |

Store primer probe mixtures at −20° C. and thaw just prior to use.

### Example 2

### RNA Extraction

The following are standard operating procedures for the extraction of RNA from viral samples using the Qiagen QIAamp® Viral RNA Mini Kit. Extreme care must be taken when working with all reagents and samples as RNA is easily degraded. Gloves must be worn at all times. This procedure was modified from the Qiagen QiAamp® Viral RNA Mini Handbook.

Prepare a solution of at least 052% sodium hypochlorite (this represents a 1:10 dilution of household or ultra bleach and is referred to herein as ‘10% bleach.’)

Clean BSC with 10% bleach, then RNase Zap and finally 70% isopropanol solution. Clean all items prior to placing in BSC with 10% bleach, then RNase Zap and finally 70% isopropanol solution, including pipettes, tips, sharps container, microcentrifuge tube rack, marker, vortexer, tubes and reagents. Change gloves.

Add 310 μL of Buffer AVE to each Carrier RNA tube. This will make a solution of 1 μg/μL. Vortex each tube for 10 seconds to make even solution. Carrier RNA solution should not be frozen and thawed more than three times, therefore break solution into convenient sized aliquots in 1.5 mL microcentrifuge tubes and store in −20° C. freezer. Expiration date of the Carrier RNA can be found on the tube label. Buffers AW1 and AW2 are received as concentrates. 100% ethanol is added to each to make the final working buffer. For the 50 reaction kit, add 25 mL of 100% ethanol to the 19 mL of AW1 concentrate to make a final total volume of 44 mL. To the 13 mL of AW2 concentrate, add 30 mL of 100% ethanol for a final volume of 43 mL.

For the 250 reaction kit, add 125 mL of 100% ethanol to 95 mL of AW1 concentrate to make 220 mL of AW1 buffer. For AW2 buffer, add 160 mL of 100% ethanol of 66 mL of concentrate for a final volume of 226 mL.

If closed tightly, all buffers can be stored at room temperature for up to one year, or kit expiration date, whichever is sooner. For ease of use, break 100% ethanol bottle into convenient sized aliquots in 15 mL conical tubes. These can be sealed tightly and stored at room temperature for one year.

Extraction of Samples Using Spin Protocol:

Clean BSC with 10% bleach, then RNase Zap and finally 70% isopropanol solution. Clean all items prior to pacing in BSC with 10% bleach, then RNase Zap and finally 70% isopropanol solution. This includes pipettes, tips, sharps container, microcentrifuge tube rack, permanent marker, vortexer, microcentrifuge, 15 mL conical tube, and reagents. Reagents include Buffer AVL, 100% ethanol, Buffer AW1, Buffer AW2, Buffer AVE and Carrier RNA solution. Change gloves.

Check the Buffer AVL. If a precipitate is seen, warm in an 80° C. water bath for not longer than 5 minutes to dissolve precipitate. For each sample extracted, label two 1.5 mL microcentrifuge tubes and one spin column with collection tube. Set up 4 additional collection tubes. Repeat for MOCK extraction control. The first 1.5 mL microcentrifuge tube will be referred to henceforth as extraction tube and the second 1.5 mL microcentrifuge tube will be referred to henceforth as the elution tube.

To determine size of extraction set, subtract one from the maximum number of tubes that will fit in the microcentrifuge. This is the maximum number of samples that can be extracted together. The final spot is for the MOCK extraction control. Shake first sample to mix. Open sample carefully and transfer 140 μL to first sample extraction tube. Close extraction tube and sample tube. Wrap sample tube cap with parafilm to seal. Repeat step for all samples in the extraction set. When all samples are sealed, wipe each down with 10% bleach and store at 4° C. To MOCK extraction control tube, add 140 μL of fresh transport media. For the rest of the procedure, handle the MOCK control before all the samples.

Use the following chart to determine how much carrier RNA to mix with buffer AVL. Extended chart can be found in the Qiagen QIAamp® Viral RNA Mini Kit Handbook.

TABLE 5

| | | Vol. |
|---|---|---|
| | Vol. | Carrier |
| No. | Buffer | RNA |
| Samples | (mL) | (μL) |
| 1 | 0.56 | 5.6 |
| 2 | 1.12 | 11.2 |
| 3 | 1.68 | 16.8 |
| 4 | 2.24 | 22.4 |
| 5 | 2.8 | 28 |
| 6 | 3.36 | 33.6 |
| 7 | 3.92. | 39.2 |
| 8 | 4.48 | 44.8 |
| 9 | 5.04 | 50.4 |
| 10 | 5.6 | 56 |
| 11 | 6.16 | 61.6 |
| 12 | 6.72 | 67.2 |
| 13 | 7.28 | 72.8 |
| 14 | 7.84 | 78.4 |
| 15 | 8.4 | 84 |
| 16 | 8.96 | 89.6 |
| 17 | 9.52 | 95.2 |
| 18 | 10.08 | 100.8 |
| 19 | 10.64 | 106.4 |
| 20 | 11.2 | 112 |
| 21 | 11.76 | 117.6 |
| 22 | 12.32 | 123.2 |
| 23 | 12.88 | 128.8 |
| 24 | 13.44 | 134.4 |

Mix AVL buffer with carrier RNA by inverting 5 times. Add 560 μL to each extraction tube, changing tips between each tub. Vortex each extraction tube 10-15 seconds. Incubate at room temperature for 10 minutes. Change gloves here and after each addition of reagent to the samples and control. Also change gloves when coming out of the BSC as well as anytime that may be necessary.

After the 10 minute incubation, add 560 μL of 100% ethanol to each extraction tube, changing tips between each tube. Vortex extraction tubes for 10-15 seconds. Briefly centrifuge to remove sample from lid. Transfer 630 μL from extraction tube into correspondingly labeled spin column. Load spin columns with collection tubes one at a time into microcentrifuge. Centrifuge spin columns for 1 minute at 6000 rcf. Remove spin columns from centrifuge one at a time. Discard collection tube and transfer spin column to a new collection tube. Transfer remaining 620 μL from extraction tube to correspondingly labeled spin column. Load spin columns with collection tubes one at a time into microcentrifuge. Centrifuge 1 minute at 6000 rcf. Remove spin columns from centrifuge one at a time. Discard collection tube and transfer spin column to a new collection tube. Add 500 μL of AW1 buffer to each sample and control. Load spin columns with collection tubes one at a time into microcentrifuge. Centrifuge tubes for 1 minute at 6,000 rcf. Remove spin columns from centrifuge one at a time. Discard collection tube and transfer spin column to a new collection tube. Add 500 μL of AW2 buffer to each sample and control. Load spin columns with collection tubes one at a time into microcentrifuge. Centrifuge tubes for 3 minutes at 20,000 rcf (max centrifuge speed.) remove spin columns from centrifuge one at a time. Discard collection tube and transfer spin column to a new collection tube.

To ensure all liquid that may compromise further processes is removed, load the spin columns with collection tubes one at a time into the microcentrifuge. Centrifuge at max speed for 1 minute to dry the spin columns. Remove spin columns from centrifuge one at a time. Discard collection tubes and transfer spin column to correspondingly labeled elution tube. Add 40 μL of room temperature AVE buffer to each sample and control. Add liquid as close to center of spin column filter as possible without touching. Incubate one minute at room temperature. Load spin columns with elution tubes into microcentrifuge one at a time. Load tubes so that the open caps are towards the center of the rotor so as to not interfere with the centrifuge lid and also to reduce the chance that the lids will break off. Centrifuge spin columns with elution tubes for 1 minute at 6,000 rcf. Remove spin columns and elution tubes from centrifuge carefully, but keep spin columns in the elution tubes. Repeat for a second elution of each sample and control.

Visually check that the correct volume of sample (80 μL) is present before discarding spin column. If incorrect volume, repeat last centrifugation step. Transfer elution tubes to 4° C. Clean all items placed in BSC with 10% bleach, then RNase Zap and finally 70% isopropanol solution before removing, clean BSC with 10% bleach, then RNase Zap and finally 70% isopropanol solution. Turn on UV light for a minimum of 15 minutes if equipped.

Extraction of Samples Using Vacuum Protocol:

Clean BSC with 10% bleach, then RNase Zap and finally 70% isopropanol solution. Clean all items prior to placing in BSC with 10% bleach, then RNase Zap and finally 70% isopropanol solution. This includes pipettes, tips, sharps container, microcentrifuge tube rack, marker, vortexer, microcentrifuge, QIAvac 24 Plus, luer plugs, manifold cap, vacuum tubing, VacConnectors and reagents. Reagents include Buffer AVL, Carrier RNA solution, 100% ethanol, Buffer AW1, Buffer AW2 and Buffer AVE. Change gloves.

For each sample to be extracted, label two 1.5 mL microcentrifuge tubes and one spin column with collection tube. Repeat set up for one MOCK extraction control tube. The first microcentrifuge tube will be referred to henceforth as extraction tube. The second microcentrifuge tube will be referred to henceforth as the elution tube. The QIAvac 24 Plus can hold 24 spin columns, so the maximum number of samples that can be extracted together in one set is 23. The final spot is for the MOCK extraction control. If the specific microcentrifuge cannot hold this many samples, refer to Handbook for guidance.

Shake first sample to mix. Open first sample carefully and transfer 140 μL to first sample extraction tube. Close extraction tube and sample tube. Wrap sample tube cap with parafilm to seal. Repeat above step for all samples in the set. When all samples are sealed, wipe each down with 10% bleach and store at 4° C. To MOCK extraction control tube, add 140 μL of fresh media. Use the chart provided with kit to determine volumes of Buffer AVL and Carrier RNA needed for the number of samples to be extracted. Mix AVL buffer with carrier RNA by inverting 5 times. Add 560 μL to each extraction tube. Vortex each extraction tube 10-15 seconds. Incubate at room temperature for 10 minutes. Change gloves here and after each addition of reagent to the samples and control. Also change gloves when coming out of the BSC as well as any time that may be necessary.

Set up the QIAvac 24 Plus by sealing one end with the cap and attaching the vacuum tubing to the opposite end. Attach a VacConnector to each opining needed in the QIAvaz 24 Plus. Attach labeled spin column to each VacConnector. Store the collection tube in a microcentrifuge tube rack until later. Any unused openings in the QIAvac 24 Plus must be sealed with a luer plug.

After 10 minute incubation, add 560 μL of 100% ethanol to each extraction tube, changing tips after each tube. Vortex each extraction tube 10-15 seconds. Turn vacuum pump on and open first spin column. Add 620 μL of liquid from first extraction tube to spin column. Verify that the label on the extraction tube corresponds with the label on the column. When liquid has been pulled completely through, repeat with remaining 630 μL of liquid. Continue until all extraction tubes have been added to their respective spin columns. Note: If at any time during vacuum extraction, liquid does not pull through the tube check all openings in manifold to make sure correctly sealed. If sealed, then revert to spin method.

After all samples have been added to columns, add 750 μL of AW1 Buffer to each spin column, changing tips between each one. Wait for all liquid to be pulled through before moving onto next step. Add 750 μL of AW2 buffer to each spin column, changing tips between each one. Wait for all liquid to be pulled through before moving on to next step. Carefully remove spin columns from manifold and place in collection tubes. To ensure all liquid that may compromise further processes is removed, load the spin columns with collection tubes one at a time into the microcentrifuge. Centrifuge at 20,000 rcf (max speed) for 1 minute to dry the spin columns.

Remove spin columns from centrifuge one at a time. Discard collection tubes and transfer spin column to correspondingly labeled elution tube. Add 40 μL of room temperature AVE buffer to each sample and control. Add liquid as close to center spin column filter as possible without touching. Incubate one minute at room temperature. Load spin columns with elution tubes into microcentrifuge one at a time. Load tubes so that the open capes are down toward the center of the rotor so as to not interfere with the centrifuge lid and also to reduce the chance that the lids will break off. Centrifuge tubes for 1 minute at 6,000 rcf. Remove spin columns and elution tubes from centrifuge carefully, but keep spin columns in the elution tubes. Repeat for a second elution of each sample and control. Visually check that the correct volume of sample (80 μL) is present before discarding spin column. If incorrect volume, repeat last centrifugation step. If volume is still incorrect, repeat [0060].

Transfer elution tubes to 4° C. Clean all items placed in BSC with 10% bleach, then RNase Zap and finally 70% isopropanol solution before removing. Clean BSC with 10% bleach, then RNase Zap and fmally 70% isopropanol solution. Turn on UV light for a minimum or 15 minutes if equipped.

Tier Two Extraction:

Any positive sample that will be re-extracted due to a positive result in Tier One testing will follow the above procedure. The only change to be made is that each positive sample is extracted with 13 replicates to ensure enough eluate to continue testing. At the end, combine the eluate (80 μL) of each replicate together into one tube before moving on to tier two testing.

### Example 3

### RRT-CRT Procedure

Tier One testing of extracted viral samples using Reverse Transcriptase-Polymerase Chain Reaction (RT-PCR). Testing is performed using the Applied Biosystems, Inc. Taqman® One-Step RT-PCR Master Mix Reagents Kit.

Calculation of Reactions Needed:

To calculate the number of reactions needed for each target, use the number of samples plus the number of mock extraction controls, plus one positive control and two No Template Controls (NTCs) per plate. If this number is under 100, add 10% to get the final number of reactions to prepare. If the number is over 100, add 15% to get final number of reactions to prepare. Fill out a coversheet with the plate layout of samples and controls as well as the number of reactions of each target to prepare and the volumes necessary.

Master Mix is Prepared with the Following Volumes per Reaction:

25 μl 2× universal Master Mix with no AMPerase UNG

1.25 μl 40× MultiScribe and RNase Inhibitor Mix

10 μl working stock primer probe mix

3.75 μl MBG water

Preparation of Master Mix: Remove working stock primer probe mixes from −20° C. freezer. Clean BSC with 10% bleach, followed by RNase Zap and finally 70% isopropanol solution. Clean all items with 10% bleach, then RNase Zap and finally 70% isopropanol solution. Remove PCR master Mix kit from refrigerator and place in BSC. Place appropriate number of 96-well plates in BSC along with MBG water and either 1.5 mL microcentrifuge tubes or 5 mL conical tubes, whichever is necessary to hold the appropriate volume of master mix. Label tubes with correct target information. Label 96-well plates if using more than one. Make sure to label plates only on the side so as to not interfere with instrument analysis. Vortex working stock primer probe mix thoroughly (5-10 seconds) before using. Vortex the 40× MultiScribe thoroughly before use; spin briefly to remove droplets from lid. Invert the 2× Universal MM to mix. Following the calculations listed on the coversheet, add each component to the appropriately labeled tubes. Change tips between each tube and between each component. Replace Taqman® One-Step RT-PCR Master Mix in refrigerator immediately after use. The working stock primer probe mixes can be stored at 4° C. if being used daily. If not using daily, store at −20° C. Place target master mixes in lab top bench cooler to keep cold while aliquoting. Work with one master mix at a time and vortex thoroughly (5-10 seconds) before using. Place the first 96-well plate on the cold block. Pipette 40 μl of target master mix into each well as designated on coversheet. Repeat with each master mix. Add 10 μl of MBG water to each of the mmNTC wells. Keeping the white backing on, place adhesive cover white side down on the plate to loosely cover. Move plate to refrigerator until transporting to sample addition area. Repeat until all plates are loaded with master mix. Clean all items in BSC and remove from BSC. Clean BSC. If equipped, turn UV light on for a least 15 minutes when finished. Remove PPE and transport coversheet and master mix filled plates to sample addition area. Transport plates in zip top bag or with gloves on.

Sample Addition of Extracted Samples:

Place all master mix loaded plates in refrigerator. Clean BSC with 10% bleach, followed by RNase Zap and finally 70% isopropanol solution. Clean all items with 10% bleach, then RNase Zap and finally 70% isopropanol solution prior to placing in BSC. Items include: 96-well cold block, calibrated pipettes and appropriate tips, sharps container, plate sealer, and microcentrifuge tube rack. Change gloves. Place MBG water and samples in BSC. Align samples in tube rack. Place first plate of master mix in the 96-well cold block in the BSC. Add 10 μl of sample to each well, change tips between each addition. Add 10 μl of MOCK extraction control to appropriate wells, changing tips between wells. Peel the white backing off the cover, being careful not to seal the positive control wells, move the sealer across the plate in one direction to cover. Be careful not to cause any wrinkles or bubbles as this may affect the reading of the wells by the instrument. Place sample loaded plate at 4° C. Repeat until all plates are loaded. Move samples to 4° C. refrigeration.

Clean all items in BSC and remove from BSC. Clean BSC. If equipped, turn on UV light for a minimum of 15 minutes. Remove PPE and transport loaded plates in zip top bags or with gloves to positive control addition area.

Addition of Positive Controls:

Place loaded plates at 4. ° C. Clean BSC and items with 10% bleach, followed by RNase Zap and 70% isopropanol solution. Items include 96-well cold block, refrigerated tube holder, plate sealer, sharps container or biohazard bag with holder, vortexer and minifuge. Change gloves. Remove positive control material from 4° C. and place in BSC. Keep in refrigerated tube holder while working with the positive controls. Place loaded plate in 96-well plate cold block. Vortex control tubes for 5-10 seconds. Add 10 μl of positive control to designated well. Use plate sealer to finish covering the plate. Repeat for all other plates. Place plate at 4° C. Clean all items in BSC, remove items from BSC and clean BSC. If equipped, turn on UV light for a minimum of 15 minutes. Remove PPE and transport plates and paperwork to thermocyclers.

Running the PCR Program:

Place sealed plates in centrifuge with plate adapters and spin for 1 minute at 1000 rpm. This ensures that all liquid moves to the bottom of the well and any air bubbles are removed. Carefully remove plates and transfer to ABI 7900HT instrument. Open SDS program. Select 96-well plate absolute quantification and the template for tier one testing. Assign detectors to each well and type in sample names.

TABLE 6

| Assay | Detector | Quencher |
|---|---|---|
| HNRPH1 | FAM | NonFluorescent |
| H5 | FAM | NonFluorescent |
| H7 | FAM | NonFluorescent |
| H9 | FAM | NonFluorescent |
| Matrix | VIC | NonFluorescent |

Check thermocycling conditions and make changes as needed. The thermocycling conditions are as follows:

50° C. for 30 minutes:

95° C. for 10 minutes:

45 cycles of 95° C. for 15 seconds and 60° C. for 1 minute.

Open instrument and place plate in instrument making sure to align A1 of plate with A1 of the plate holder. Place compression pad on top of adhesive cover making sure that the grey side is facing down towards the wells. Start run. When prompted to save file, click yes and give file a unique name.

Analyzing Results:

At end of run, a box will pop up reading run completed successfully. This confirms the instrument did not fault during the program. Click OK. Click the small Green arrow icon on toolbar. Set the threshold to manual and assign values of “0.02” for the H5/M assay and 0.05 for HNRPH1, H7/M and H9/M assays. Apply values to both detectors of the multiplexed assays. Click OK. Click the large Green arrow to analyze results. Click on the corner between A and 1 on the plate layout to be able to view results of the entire plate. Select results tab to view the amplification plot. Change detectors in lower left corner of amplification plot. Print amplification plots by individual detectors using print icon. Print Ct values for each detector. Plate information can also be printed with the printer manager. The multicomponent view can also be printed, but note that it can only be printed for individual wells.

Possible Sample Results:

Positive Control:

Positive result—master mix is okay. All sample results can be accepted. Negative result—master mix is not working correctly. New master mix is needed and all samples and controls need to be retested. Check that Taqman® One-Step RT-PCR Master Mix kit is not expired and that control material is satisfactory.

No Template Controls (NTCs):

Negative result—master mix is not contaminated and all results are valid. Positive result—master mix is contaminated. All positive samples need to be retested with new master mix to determine if positive result is from positive sample or contamination.

HNRPH1:

Positive result—extraction procedure worked for that sample and there are not inhibitors present in the sample. Negative result—RT-PCR is inhibited or extraction failed. Dilute inhibited samples 1:2 and 1:4 and rerun these dilutions on fresh master mix for all assays. If HNRPH1 still does not mix, re-extract the sample and retest for all assays.

BIAD M3:

Negative result—no influenza present in sample. Positive result: Influenza A is present in sample. Tier two testing may be required of this sample depending on the results of the H5 assays.

H5, H7 and H9:

Negative results—these HA subtypes of Influenza A are not present in the sample. Positive result—The specific HA subtype of Influenza A that was being targeted for is present. If H5 is detected proceed with re-extraction of sample and Tier Two testing of re-extracts.

### Example 4

### Pyrosequencing Control Preparation

Ordering Primers:

Primers are ordered from Integrated DNA Technologies. Primers are received lyophilized and reconstituted as directed to form stock solutions.

Preparation of Control Template Mix:

Dilute 4 μL of 10 μM Control Template Primer with 196 μL MQ-H20. Label with assay name, preparers initial and date prepared. Store Template mixtures at −20° C. and thaw just prior to use.

Preparation of Control No VacuumPrep Mix:

Combine the Following to Prepare Mix:

2.5 μL of 10 μM Control Template Primer;

7.5 μL of 10 μM Control SQ Primer;

40 μL 1× Annealing Buffer.

Heat the tube at 80° C. for 2 minutes. Move to room temperature and let cool to room temperature, approximately 10 minutes. Label with assay name, date prepared and preparers initials. Store at −20° C. and thaw just prior to use.

### Example 5

### Pyrosequencing

This Example contains standard procedures for two tier testing of extracted vial samples using RT-PCR performed using Qiagen One-Step RT-PCR kit.

Determine amount of master mix needed. Each tier one positive sample will need 90 tier two RT-PCR reactions that will then be sequenced. The following volumes are needed per reaction.

TABLE 7

| Reagent | Volume (1 × rxn) | Volume (×100rxs) |
|---|---|---|
| 5x buffer* | 4 μL | 400 μL |
| 10 mM dNTPs* | 0.8 μL | 80 μL |
| Enzyme Mix* | 0.8 μL | 80 μL |
| 40 U/μL RNase | 0.1 μL | 10 μL |
| Inhibitor | | |
| MBG Water* | 1.1 μL | 110 μL |
| Extracted Sample | 10 μL | 1000 μL |
| Total | 16.8 μL | 1680 μL |

*included in Qiagen One Step RT-PCR Kit

Primer Addition and Master Mix Preparation:

Remove working stock primer mixes (prepared according to Primer Probe Prep Example 1) from freezer. Clean BSC with 10% bleach, followed by RNase Zap and finally 70% isopropanol solution. Clean all items with 10% bleach, then RNase Zap and finally 70% isopropanol solution prior to placing in BSC. Items include calibrated pipettes with appropriate tips, 96-well cold block, refrigerated microcentrifuge tube holder vortexer, minifuge and marker. Change gloves. Place one 96-well plate per positive tier one sample and the Qiagen One-Step RT-PCT kit in the BSC. Place all tubes containing enzymes from kit immediately in the refrigerated tube holder. Place correct size tube (1.5 mL or 5 mL in BSC, as well as all primer mixes. Label 2 mL tubes and 96-well plates with sample numbers. Make sure to label plates only on the side so as to not interfere with instrument analysis. Vortex and briefly spin down thawed pyrosequencing target primer mixer. Add 3.2 μL of each pyrosequencing target primer mixture to their assigned wells as indicated on form 2-PCR Plate Layout. Changes tips between each well. Sample is tested in duplicate for each pyrosequencing target. Place adhesive cover white side down on plate without removing backing to loosely cover the plate. Place plate at 4° C.

If Sample Addition room is available, clean all items placed in BSC before removing. Clean BSC with 10% bleach, then RNase Zap and finally 70% isopropanol solution. Remove PPE and transport master mix and 96-well plates containing primer mixes to sample addition area. If continuing work in same laboratory, return all master mix components to freezer. Clean BSC with 10% bleach, then RNase Zap and finally 70% isopropanol solution.

Sample Addition of Extracted Tier Two Samples:

Add appropriate amount of re-extracted sample to 2 mL tube containing master mix. Vortex master mix 10-15 seconds. Place one 96-well plate containing pyrosequencing primer mixes in the cold block. Pipette 16.8 μL of master mix into wells changing tips between each addition. Remove white backing from adhesive plate cover and seal plate in one motion across the wells making sure not to wrinkle the cover or form bubbles. Remove sample loaded 96-well plate and place at 4° C. Repeat for each re-extracted sample until all plates have been loaded. Clean all items in hood and BSC with 10% bleach, then RNase Zap and finally 70% isopropanol solution, turn on UV light for a minimum of 15 minutes if equipped. Remove PPE and transport sample loaded plates to RT-PCR area.

RT-PCR:

Place sealed, sample loaded plates in centrifuge with plate adapter and spin for 1 minute at 1000 rpm to bring everything to the bottom of the wells with no bubbles. Place sealed, sample loaded plates in thermocycler. If required by manufacturer, add compression pad or other device on top of plate to prevent evaporation.

Program Thermocycler to Run with the Following Condition:

50° C. for 30 minutes;

95° C. for 15 minutes;

45 cycles of 94° C. for 45 seconds, 55° C. for 45 seconds, 72° C. for 1 minute;

72° C. for 10 minutes;

4° C. for ∞.

Pyrosequencing:

Turn on Pyromark instrument 1-2 hours before use. Turn on 80° C. heat plate so it has time to get up to temperature. Remove binding buffer, annealing buffer, sepharose beads, PyoGold Reagents and wash buffer from 4° C. and equilibrate to room temperature. Remove control Template from freezer and thaw completely before use. Remove 5 μM sequencing primers from freezer and allow to thaw just before use. Refer to form 2 for calculations and plate orientation. Prepare binding solution by mixing 40 μL of binding buffer, 3 μL sepharose beads (do not vortex) and 20 μL of water per reaction. Make one extra reaction to compensate for loss during pipetting.

Carefully remove adhesive cover from PCR plate. Add 20 μL control template mix to the PCR plate to serve as a VacuumPrep Control. Vortex binding solution thoroughly (10-15 seconds) and add 60 μL to each PCR reaction well. Change tips between each well. Briefly vortex binding solution after every row to eliminate settling of beads.

Seal plate again (with new adhesive cover if necessary) and agitate at 1400 rpm for 10-60 minutes before moving on to vacuum step. Do not remove from shaker until following steps are completed. Pipette 36 μL of annealing buffer to each target well of a PSQ 96 low walled plate. Using plate layout to ensure proper sequencing (SQ) plate orientation with the PCR plate, add 4 μL of defined 5 μL sequencing primer to each well. Pipette up and down to mix primer into buffer. Add 38 μL annealing buffer and 2 μL of control SQ primer to “VaccumPrep Control” well. Add 32 μL annealing buffer and 8 μL of prepared control No VacuumPrep Mix to the “No VacuumPrep Control” well. Set up troughs of the vacuum prep station. Fill each of the following troughs with approximately 180 mL of solution: priming water (high purity), 70% ethanol solution, denaturation solution (120 mL), wash buffer, and wash water (high purity.) Note: Wash buffer liquid level should be slightly higher than denaturing solution liquid to ensure complete removal of denaturing solution from VacuumPrep tool.

No more than 3 minutes before vacuum preparation, remove PCR plate from mixer and carefully remove adhesive cover. Discard cover. Turn on the vacuum of the prep station. Wash the probes of the tool by placing in the priming water trough for approximately 20 seconds. Verify that a correct vacuum has been obtained by ensuring the arm of the vacuum gauge has moved beyond the red zone. Tilt the tool in your hand to remove all liquid that is in the hand held section before moving on.

Capture the beads containing immobilized templates on the filter probes by slowly lowering the VacuumPrep Tool into the PCR plate. Ensure that the liquid had been aspirated evenly from all wells and that all beads have been captured onto the probes. Transfer tool to 70% ethanol solution and let liquid pull through probes for 5-10 seconds before removing. Tilt the tool in your hand to remove all liquid that is in the hand held section before moving on. Repeat last step for denaturation solution and finally wash buffer troughs. After wash buffer, turn vacuum off and disconnect tool from the station. Carefully place tool in annealing plate ensuring that the A1 indicator on tool is aligned with well A1 of sequencing plate. Move the probes in a circular motion on bottom of wells of annealing plate to displace beads into the annealing buffer. Remove tool from plate.

If sequencing more than one plate, transfer annealing plate to refrigerator until ready to sequence. T clean VacuumPrep tool, shake in high purity water then turn vacuum on. Clean tool by placing in last water trough and allowing liquid to pull through for 5-10 seconds. Repeat steps for all plates. Make sure to clean vacuum probes well between each plate. Place annealing plate on 80° C. hot plate for 2 minutes. Allow plate to cool to room temperature, about 10 minutes.

Set up a new Biotage SQA sequencing run. Refer to pyrosequencing parameter information to obtain calculated reagent volumes. Fill reagent cartridge with high purity water. Press firmly on top of each opening and verify that a straight, steady stream exits through needles on bottom of cartridge. Empty water from cartridge and repeat test. Discard water and shake firmly to dry out cartridge, ensuring no liquid is on the outside of the needles. Reconstitute enzyme and substrate of PyroGold kit in 620 μL of high purity water. Do not vortex. Swirl tubes to mix and incubate at room temperature for 10 minutes to ensure all reagent has dissolved. Add PyroGold reagents (Enzyme (E), Substrate (S), dATP (A), dGTP (G), dCTP (C) dTTP (T)) to appropriate slots in reagent cartridge referring to run calculated reagent volumes.

TABLE 8

| | G | |
|---|---|---|
| C | | T TTT |
| S | A | E |

Place SQ plate in pyrosequencer, close plate lid. Place the cartridge in pyrosequencer, click the cartridge lid into place. Close pyrosequencer cover and start run. At end of run, discard plate and clean reagent cartridge by forcing high purity water through the probes.

Analysis of Sequences:

At the end of the sequencing run, analyze entire plate. The wells will be colored on the screen depending on the sequencing results. Blue wells pass, yellow wells need the sequence to be checked, and red wells fail.

Print out sequences. Open PyroMark ID software. Import all sequences from latest SQA run into software. Select the S.A.F.E. library from the drop down menu. Add this library to all sequences. Analyze sequences. Print out library results.

Each library result will show the best possible matches. If results indicate avian, then the H5 present in the initial sample is to be reported as “Avian”. If results indicate any site is “Human”, then the H5 present in the initial sample is to be reported as potential human threat.

### Example 6

Primers and Probes for RRT-PCR were designed against the HA and the M gene segments. Sequences available through the Influenza Virus Resource (NCBI) were aligned and analyzed to allow for incorporation of mixed bases. Specific HA subtype sequences were used for designing the HA primer/probe set and all Influenza A sequences were used for designing the M primer/probe set. Each HA subtype assays is multiplexed with the M RRT-PCR assay, i.e. H5/M refers to a multiplexed assay to detect the H5 subtype and the same M target in all assays.

The limit of detection (LOD) for the RRT-PCR assays was determined by using a 10-fold dilution series of purified viral RNA. The lowest concentration yielding 3/3 positive indications was deemed the broad range LOD. This concentration was used as the starting point for a series of five 2-fold serial dilutions. Again, the lowest concentration yielding 3/3 positive indications was determined to be the LOD. The quantification of total Influenza RNA was based on hemagglutination titers of allantoic fluid used to purify the RNA. The H5N1 RNA used for LOD determination was purified from 400 μl of allantoic fluid with hemagglutination titers of 20 HA units/μl. The total purified RNA was resuspended in 100 μl and was defined as RNA representing 80 HA units/μl. The H7 RNA represents 20 HA units/μl and the H9 RNA represents 40 HA units/μl.

To determine the specificity of the RRT-PCT assays, each was challenged with five HA subtypes (H1, H3, H5, H7 and H9) in clean and dirty matrices. ‘Clean’ matrices are mock extractions of water and ‘dirty’ matrices are extractions of chicken throat swabs. H5, H7 and H9 assays only detected their respective subtypes, while the M assay detected each subtype 100% of the time. The H7/M and the H9/M assays failed to detect the M target from H5N1 RNA only when the RNA was very dilute (2×10⁶ fold dilution); matrix type, clean or dirty, had no effect. These date show that the multiplexed RRT-PCR assays are specific, discriminating between subtypes.

False negative and positive rates were determined by challenging the assays with clean and dirty matrices which were either spiked with appropriate target or unspiked. Spiked samples were used to determine the false negative rate using the following equation: 100%*[1-(true negative/known negative)]. Unspiked samples were used to determine the false positive rate using the following equation: 100%*[1-(true positive/known positive)]. Forty replicates of each experimental scenario were tested to allow for determining an approximately 10% failure rate with greater than 95% confidence. All three multiplexed RRT-PCR assays resulted in 0% false positive rates and less than 10% false negative rates in both clean and dirty matrices. This data shows the high level of accuracy obtained with the assays.

Pyrosequencing assays were designed to detect codons encoding the 52 amino acid sites defined as human or avian influenza virus signatures. Because some signatures were detectable within a single sequencing read, 45 assays accounted for the 52 target sites. Each of the 45 pyrosequencing reactions were tested for accuracy by analyzing H5N1 RNA spiked into extract from clean and dirty matrices. False negative rates were calculated as with the RRT-PCR accuracy determination. Of the 45 assays, 33 resulted in zero false negatives in the clean matrix and 12 resulted in less than or equal to 10% failure rate in the clean matrix. The false negative rates were slightly higher in the dirty matrix, less than or equal to 12.5%.

There has therefore been provided a method of detecting of avian influenza viruses and monitoring emerging mutations which would make the viruses capable of causing a pandemic.

Having described the invention in detail, those skilled in the art will appreciate that modifications may be made of the invention without departing from its' spirit and scope. The main focus of the illustrative embodiments has been to multiplex the M and H5 RRT-PCR reactions for simultaneous detection of general Influenza A, and more specifically H5N1. However, detection of other highly pathogenic strains, for example H7 and H9 would also be valuable. Successful multiplex RRT-PCR reaction have been accomplished in the prior art with up to four different primer/probe sets using FAM, NED, VIC and TET. These four fluorophores have been used in efforts to multiplex M H5, H7, and H9 detection. The present method may also serve as a model for establishing surveillance of other emerging RNA viral threats. Therefore, it is not intended that the scope of the invention be limited to the specific embodiments described. Rather, it is intended that the appended claims and their equivalents determine the scope of the invention.

What is claimed is:

1. A method comprising sequencing RRT-PCR with pyrosequencing to identify high pathogenic avian strains and then detect mutations in the high pathogenic avian strains that render the virus more infective to humans.

2. A method for detecting emerging pandemic influenza, the method comprising:
a) performing RRT-PCR to simultaneously detect multiple Influenza A virus subtypes and H5 strains; and
b) pyrosequencing targeted regions of gene segments of the H5 strain to determine if critical human virulence signatures are present.

3. The method of claim 1 further including isolating virus RNA prior to performing RRT-PCR.

4. The method of claim 1 further including amplifying gene segments prior to pyrosequencing.

5. The method of claim 1 further including conducting mutation analyses of the critical human virulence signatures.

6. A kit comprising materials required to perform the method of claim 2.

7. A method for detecting emerging pandemic influenza, the method comprising:
a) performing RRT-PCR to simultaneously detect multiple Influenza A virus subtypes and H5 strains;
b) amplifying gene segments of the H5 strain; and
c) pyrosequencing targeted regions of the gene segments of the H5 strain to determine if critical human virulence signatures are present.

8. The method of claim 7 further including isolating virus RNA prior to performing RRT-PCR.

9. The method of claim 7 further including conducting mutation analyses of the critical human virulence signatures.

10. A kit comprising materials necessary for performing the method of claim 7.

11. A method for detecting emerging pandemic influenza, the method comprising:
a) isolating virus RNA;
b) performing RRT-PCR to simultaneously detect multiple Influenza A virus subtypes to detect H5N1 strain;
c) amplifying gene segments of the H5N1 strain; and
d) pyrosequencing targeted regions of the gene segments of the H5N1 strain to determine if critical human virulence signatures are present; and
e) conducting mutation analyses of the critical human virulence signatures.

12. A kit comprising the materials required to perform the method of claim 11.
